(19) United States
(12) Patent Application Publication
(10) Pub. No.: US 2025/0115646 A1
(43) Pub. Date: Apr. 10, 2025

(54) METHOD AND DEVICE FOR PAIN MODULATION BY OPTICAL ACTIVATION OF NEURONS AND OTHER CELLS

(71) Applicant: Samarendra Kumar Mohanty, Arlington, TX (US)

(72) Inventor: Samarendra Kumar Mohanty, Arlington, TX (US)

(21) Appl. No.: 18/887,005

(22) Filed: Sep. 16, 2024

Related U.S. Application Data
(63) Continuation of application No. 15/822,185, filed on Nov. 26, 2017, now abandoned.
(60) Provisional application No. 62/426,402, filed on Nov. 25, 2016.

(51) Int. Cl. C07K 14/435; A61K 9/00; A61K 41/00; A61K 47/64; C12N 15/86

(52) U.S. Cl. CPC C07K 14/435; A61K 9/0002; A61K 41/0042; A61K 41/0057; A61K 47/6425; C12N 15/86

(57) ABSTRACT

This invention, in one aspect, relates generally to methods for optically modulating pain in animals and human. The invention provides method for the use of opsin for modulating pain, wherein optical stimulation of specific neurons and/or other cells in targeted regions of the nervous system sensitized by opsin, using genetic technologies, leads to significant reduction of pain perception to noxious stimuli. Further, the invention provides a method for inhibition of pain without use of exogenous opsin, wherein visual stimulation of eye (having endogenous opsin) is carried out. The invention also includes device(s) for controlled modulation neural and/or cellular activities in brain, eye and peripheral nervous system in order to treat different forms of chronic pain.

### CROSS-REFERENCE

This application claims the benefit of U.S. provisional application No. 62/426,402 filed Nov. 25, 2016, which application is incorporated herein by reference.

### STATEMENT REGARDING FEDERALLY SPONSORED RESEARCH OR DEVELOPMENT

This invention was made with private funding by NanoScope Technologies, LLC. The government has no rights in the invention.

### DESCRIPTION OF THE TEXT FILE SUBMITTED ELECTRONICALLY

The contents of the text field submitted electronically herewith are incorporated herein by reference in their entirety: A computer readable format copy of the Sequence Listing (file name: SAMAR2017A_SL.txt, date recorded: Nov. 25, 2017, file size 289 kilobytes).

### FIELD OF THE INVENTION

This invention relates generally to methods for optically modulating pain in animals and human. More specifically, the invention provides method for the use of opsin for modulating pain, wherein optical stimulation of specific neurons and/or other cells in targeted regions of the nervous system sensitized by opsin, using genetic technologies, leads to significant reduction of pain perception to noxious stimuli. Further, the invention relates to a method for inhibition of pain without use of exogenous opsin, wherein visual stimulation of eye (having endogenous opsin) is carried out. The invention also provides device(s) for controlled modulation neural and cellular activities in brain, eye or peripheral nervous system in order to treat different forms of chronic pain.

### BACKGROUND OF THE INVENTION

Modulation of neural activities by electrical and other means has enabled modulation of physiological functions. Opsins (light-sensitive ion-channel proteins) in combination with light have been used for modulation of neural activity.

Current pain relief approaches range from pharmaceutical to electrical or magnetic based deep brain stimulation and neuron ablation therapy, but there is no single treatment to alleviate different types of pain including sever migraine, phantom pain, chronic back pain and pain due to rheumatoid arthritis. In past, investigations of cellular mechanisms for physiological and pathological pain are focused mainly on the periphery and the spinal dorsal horn, considering potential less central side effects of drugs. However, despite progress achieved over many years, many forms of chronic pain are still resistant to conventional analgesics and drugs. Although there are a range of existing clinical options, a lot of patients don't respond to any of them, resulting in opioid dependence. Overprescription of opioids for chronic pain is severely costing many lives.

Different regions of central and peripheral nervous systems and specific cells are involved in pain perception, and its processing. For example, thalamus receives projections from multiple ascending pain pathways and is involved in processing of the nociceptive information before relaying the information to various cortical regions. The thalamic nuclei are involved in the sensory discriminative and affective motivational components of pain. The thalamic neurons project to the dorsal hom of spinal cord and modulates ascending nociceptive information. Changes in the gene expression, biochemistry, thalamic blood flow and responsiveness of thalamic neurons have been shown In the animal models for pain, which suggest role of the thalamus in modulating pain. Recent work has concentrated in testing the effect of electrical stimulation of the Thalamus, as a strategy for pain control. However, electrical pulses delivered via electrodes implanted in the deep brain (1, 2) lack the specificity in stimulating particular group of neurons, and thus the precise involvement of specific Thalamic neurons in pain modulation remains to be determined.

The advent of neuronal stimulation using optogenetics has enabled highly selective activation of specific as well as several types of neurons with millisecond temporal precision (3, 4) using light of different wavelengths. As compared to electrical stimulation, optogenetics is more specific and multiple types of neurons can be selectively targeted within the same region of the nervous device (5, 6). This light-assisted method of cellular stimulation eliminates the highly challenging requirement of placing electrodes in brain nuclei with relatively homogeneous group of neurons. This characteristic has also led to the emerging of optogenetics as a valuable expenmental tool and a promising approach for studying a variety of neurological disorders, such as blindness (7-10), drug-addiction (11, 12), conditioned fear (13), and Parkinsonian symptoms (14) in animal models, Channelrodopsin-2 (ChR2), a non-selective cation channel, is the most commonly used opsin for depolarization of neurons (4, 15).

Optogenetic modulation provides high cellular-specificity by introduction of light-activated molecular channels (opsins) by genetic targeting in a promoter-specific manner (4, 16, 17). In order to achieve optogenetic stimulation of specific neurons, the cells are typically transfected by a virus to express opsin, which gets activated, thus depolanzing the opsin-expressing cells when illuminated by light of specific band of light, characteristics of the opsin. For example, cells expressing ChR2 are sensitive to blue light.

To the extent that any specific disclosure in the aforementioned references or other literature may be considered to anticipate any genenc aspect of the present invention, the disclosure of the present invention should be understood to include a proviso or provisos that exclude of disclaim any such species that were previously disclosed. The aspects of the present invention, which are not anticipated by the disclosure of such literature, are also nonobvious from the disclosure of these publications, due at least in part to the unexpectedly superior results disclosed or alleged herein.

### SUMMARY OF THE INVENTION

in order to meet the challenges, the inventor has created several molecules and methods for optically modulating pain in animals and human. The invention also provides device(s) for controlled modulation neural activities in brain and eye in order to treat different forms of chronic pain.

in one aspect, the disclosure provides Bioluminescent Bandwidth engineered Opsin-1 (B2EO-1, SEQ ID NO: 2) protein that, when expressed on cell membrane, excites the targeted celis upon activation by external (active) light illumination or by intrinsic bioluminescence emitted from the targeted cells themselves in presence of injected co-factors (e.g. furimazine, or analogs).

In another aspect, the disclosure provides Bioluminescent Bandwidth engineered Opsin (B2EO-2, SEQ ID NO: 3) that, when expressed on cell membrane, silences the targeted cells upon activation by external (active) light illumination or by intrinsic bioluminescence emitted from the targeted cells themselves in presence of injected co-factors.

The delivery of the B2EO opsin-genes to targeted region(s) of brain or peripheral nervous system is carried out by injection of virus carrying promoter-B2EO-1/2 or by other physical/chemical methods.

In one aspect, the disclosed invention provides method for the use of opsin for modulating pain, wherein optical stimulation of specific neurons in targeted regions of the nervous device sensitized by opsin, using genetic technologies, leads to significant reduction of pain perception to noxious stimuli.

According to another aspect of the invention, the invention includes a method for inhibition of pain without use of exogenous opsin, wherein visual stimulation of eye (having endogenous opsin) is carried out using specific wavelength and frequency of visible light.

Advantages of the present approach is the fact that it targets specific neurons and cells in targeted regions of the central and peripheral nervous systems, unlike global stimulation by existing electrical/magnetic approaches; thus generating better efficacy for modulating pain.

The present inventor investigated if optogenetic stimulation of excitatory neurons in different regions of Thalamus can modulate pain in animal model. The results presented herein show efficient pain inhibition in awakened mice subjected to optogenetic stimulation of ChR2-sensitized excitatory neurons in Thalamus. The results also demonstrated that the pain modulation is dependent on the frequency and intensity of optogenetic stimulation.

In another aspect, the present inventor demonstrated that use of different Bioluminescent Bandwidth engineered Opsins (B2EO-1, 2) leads to passive modulation of the cells by their activity-induced emission of bioluminescence, which when applied to neurons, fibroblasts, astrocytes/glia, immune cells, keratinocytes and/or vascular endothelial cells of central/peripheral nervous system leads to down-regulation of release of neurotransmitters, or release of pro-inflammatory cytokine(s); or up-regulation of endorphins and anti-inflammatory agents leading to pain inhibition.

According to yet another aspect of the invention, method of efficient pain inhibition uses eyes as optical windows to the pain perception circuits of the brain. In this method, suitable wavelength and mode of light stimulation of eye led to significant inhibition of pain in awakened animals.

In another aspect, the invention provides device(s) for controlled modulation of neural and other cellular activities in brain, eye and peripheral nervous systems in order to treat different forms of chronic pain.

In another embodiment, the present invention includes methods and uses of the B2EO-1, or B2EO-2 for treatment of pain: wherein the use comprises delivery of the B2EO-genes to different cells of different organs by either chemical, viral or physical transduction method; wherein activation of B2EO is achieved upon illumination of external light or intrinsic bioluminescence (in presence of co-factor); and wherein an effect is measured by an electrophysiology or other functional and behavioral analysis.

In one aspect, the nucleic acid has at least one of 75%, 85%, 95% or 100% identity to SEQ ID NO: 2, or 3. In another embodiment, the invention includes a vector comprising the nucleic acid having 75%, 85%, 95% or 100% identity to at least one of SEQ ID NO: 2 or 3. In one aspect, the vector is selected from an adenovirus, adeno-associated virus or lentivirus vector. In another embodiment, the present invention includes a method of treating pain composing administering to a patient in need thereof a vector comprising the nucleic acid having 75%, 85%, 95% or 100% identity to at least one of SEQ ID NO: 2 or 3.

Details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears.

Tables 1-2 show Amino acid sequences of Bioluminescent Bandwidth engineered Opsins (B2EO): B2EO-1, and B2EO-2. Table-3 shows the DNA sequences of promoter (CAG) used upstream of B2EO-sequences for targeting specific cells as an example.

FIG. 1A illustrates Schematics of pain descending pathway during electrical stimulation of nervous device. Electrical stimulation is not specific in stimulating different types of neurons and thus both inhibitory and excitatory neurons will be stimulated. FIG. 1B shows that optogenetic stimulation, only cell-specific (inhibitory/excitatory) neurons can be modulated in targeted Thalamus and other brain regions. This leads to controlled modulation of pain by selective enhancement of the neural circuitries (e.g. Thalamus-PAG-Dorsal horn). Increased activity (+) and decreased activity (−).

FIG. 2A shows an optical fiber stub implanted in to the brain for the delivery of light. 2000: Optical fiber stub. FIG. 2B shows the stimulating light source (LED) connected to the implanted optical fiber stub act as Optical neural stimulator. 2010: LED coupled with fiber. 2020: Power supply for the LED.

FIG. 3A shows optimization of parameters for optogenetic stimulation of Thalamus. Monte Carlo simulation of 470 nm light propagation in two-layered cortex in Natural log of intensity (in W/cm²) for 5 mW of power emanating from the optical fiber, delivered by optical fiber with NA=0.3. The light propagation in Natural log of intensity (in W/cm²) for 50 mW of power emanating from the optical fiber is shown in FIG. 3B.

FIG. 4A shows behavioral assay of pain modulation by optogenetic stimulation of Thalamus. Experimental scheme for evaluating modulation of Formalin induced pain by optical stimulation. FIG. 4B shows Pain scoring (weighted average of lifting, licking of injected paw) as a function of time after formalin injection. No Light, No Formalin: Absence of stimulation light and no formalin injection. Light alone: Pre-formalin pain score in presence of light stimulation of Thalamus, Formalin alone: pain score of same mouse in post-formalin condition in absence of light stimulation.

FIG. 5A shows the behavioral assay of pain modulation by optogenetic stimulation of Thalamus. Pain score: weighted average of lifting (weight=1), licking (weight=2) of injected paw. Pain scoring as a function of time after formalin injection for Mouse-1.

Formalin: post-formalin condition in absence of light stimulation.
Formalin+Light: post-formalin condition in presence of light stimulation of Thalamus.

FIG. 5B shows the behavioral assay of pain modulation by optogenetic stimulation of Thalamus. Pain score: weighted average of lifting (weight=1), licking (weight=2) of injected paw. Pain sconng as a function of time after formalin injection for Mouse-2.

Formalin: post-formalin condition in absence of light stimulation.
Formalin+Light: post-formalin condition in presence of light stimulation of Thalamus.

FIG. 6A shows the histology of mice brain that have undergone optogenetic stimulation of Thalamus showing location of optical fiber (dotted line). Coronal section of Mice 1 showing stimulation of Ventral anterior-lateral complex (VAL), ventral posterolateral nucleus (VPL) and ventral medial nucleus (VM) of thalamus. FIG. 6B shows the histology of mice brain that have undergone optogenetic stimulation of Thalamus showing location of optical fiber (dotted line). Coronal section of Mice 2 showing stimulation of Ventral medial nucleus (VM) and parvicellular part of ventral posteromedial nucleus (VPMpc) of thalamus. Scale bar: 1 mm.

FIG. 7A shows the coronal section of Mice 2 showing location of optical fiber (dotted line) used for stimulation of Ventral medial nucleus (VM) and parvicellular part of ventral posteromedial nucleus (VPMpc) of thalamus. FIG. 7B illustrates the analysis of ChR2-YFP expression near the tip of the inserted fiber stub in the mouse brain. Confocal fluorescence image of ChR2-YFP expression in the Coronal section of mouse brain. YFP Fluorescence near the tip of the inserted fiber stub in Mice 2 confirms ChR2 expression in targeted regions (VM, VPMpc) of thalamus.

FIG. 8A shows the schematic design of delivery of light sensitizing agents and active optical modulation of cellular activities. 8000 B2EO; 8010: Cannula; 8020: Delivery device; 8030: Targeted CNS/PNS region; 8040: Optical stimulation source; 8050: Light guide; 8060: Wireless control for optical stimulation source (8040), FIG. 8B shows the flow chart for optical modulation of cellular activities in CNS/PNS for pain inhibition.

FIG. 9 shows the mechanism for B2EO based passive modulation of cellular activities in CNS/PNS for pain inhibition. Once chronic pain sensation persists, B2EO is injected to target organ to express in targeted cells. Co-factor is injected after B2EO expression to result in bioluminescence, which modulates activities of targeted B2EO expressing cells in order to attenuate pain sensation. Re-injection or uptake of co-factor is carried out at intervals to mitigate recurrence of pain sensation.

FIG. 10A shows the map of plasmid encoding Bioluminescent Bandwidth engineered Opsin (B2EO). FIG. 10B shows the gel electrophoresis of B2EO after restriction digest using BamH/Sall. FIG. 10C shows the fluorescence image of B2EO-expressing cells. FIG. 10D shows the representative inward current in B2EO-expressing cells in response to a light (average intensity: 0.09 mW/mm²) pulse (in trace 1) measured by Patch-clamp electrophysiology. Recurrence of inward photocurrent is evident in traces 2, 3 & 4 even after switching off the initial light pulse (during trace 1).

FIG. 11A shows the B2EO based passive modulation of cellular activities in CNS/PNS for pain inhibition. 1100: Delivery of Bioluminescent Bandwidth engineered Opsin (B2EO) carried out by injection 1110: delivery of co-factor carried out later either by injection or oral uptake. FIG. 11B shows the flow chart for 82EO based passive modulation of cellular activities in CNS/PNS for pain inhibition.

FIG. 12A shows the set up for optical stimulation of eye to evaluate effect on pain modulation. Counter-propagating strobe lights applied just after injecting formalin to right hind paw of a mouse. Though swelling and redness observed in the injected paw (encircled), the mouse was seen to walk naturally inside the cage. FIG. 12B shows that at approximately 40 min after injecting formalin to nght hind paw of the mouse, it was observed to be standing using two hind paws and explore the environment. FIG. 12C shows the spectrum of the strobe light used for modulating pain.

FIG. 13A shows the behavioral assay of pain modulation by optical stimulation of eyes of wild-type mice. Pain score: weighted average of lifting (weight=1), licking (weight=2) of injected paw. Pain scoring as a function of time after formalin injection for Mouse-1 in presence of visual stimulation. FIG. 13B shows the pain scoring as a function of time after formalin injection for Mouse-2 in presence of visual stimulation.

FIG. 14A shows the macro level depiction of active optical stimulation of eye for pain inhibition. 1400: Light source; 1410: Eye with intact photosensitivity of retina (rods/cones, intrinsically-photosensitive RGCs); 1420: Non-injured optic nerve. FIG. 148 shows the detailed depiction of circuitry for optically-stimulated eye induced modulated brain activities for pain inhibition. 1430: Light projecting goggles; 1440: power supply and controller; 1450: LGN of Thalamus.

FIG. 15A shows the behavioral assay of pain modulation by optical stimulation of Thy1-ChR2 sensitized eye. Pain score: weighted average of lifting (weight=1), licking (weight=2) of injected paw. Pain scoring as a function of time after formalin injection for Mouse-1 in presence of visual stimulation. FIG. 15B shows the pain scoring as a function of time after formalin injection for Mouse-2 in presence of visual stimulation.

### DETAILED DESCRIPTION OF THE INVENTION

The present disclosure will now be described more fully hereinafter with reference to the accompanying drawings, in which some exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Pain is a major world-wide health issue leading to severe impairment of normal psychological and physical conditions of the patients. While neuropathic pain (18, 19) is caused by damage to the nervous device, nociceptive pain (20) is caused by stimulation of peripheral nerve fibers that respond to severe harmful stimuli. Under persistent activation, nociceptive transmission to the dorsal horn (21, 22) may induce neuro-pathological changes that lower the threshold (allodynia) for pain signals to be transmitted to the sensory cortex, and thus enhances pain sensitivity. In addition, it may create non-nociceptive nerve fibers to respond to pain signals leading to enhanced pain sensation in response to noxious stimuli (hyperalgesia) (22). This process is difficult to reverse or eradicate in chronic pain, once established Chronic pain is estimated to affect 25 percent of Americans and account for more than 20 percent of all physician office visits (23). Sustained inhibition of many neuropathies and most idiopathic chronic pain is rarely achieved and management of chronic pain has emerged as a significant challenge. Therefore, there is an intense need for development of new therapeutic strategies for managing chronic pain, and improve quality of life.

Chronic pain can occur in response to long-term changes in plasticity along sensory pathways in peripheral nociceptors, spinal dorsal horn, sub-cortical areas including Thalamus as well as cortical areas that are involved in the processing of painful information (24). The thalamus receives projections from multiple ascending pain pathways and is involved in processing of the nociceptive information before relaying the information to various cortical regions. For example ventrobasal complex (VB) consisting of the Ventral posteromedial nucleus (VPM) and the Ventral posterolateral nucleus (VPL), is a relay nucleus of the thalamus for nociceptive stimuli received from nociceptive nerves in sensory discriminative component and the modulation of that stimuli to the primary somatosensory cortex. Similarly, the intralaminar nuclei is involved in affective-motivational component.

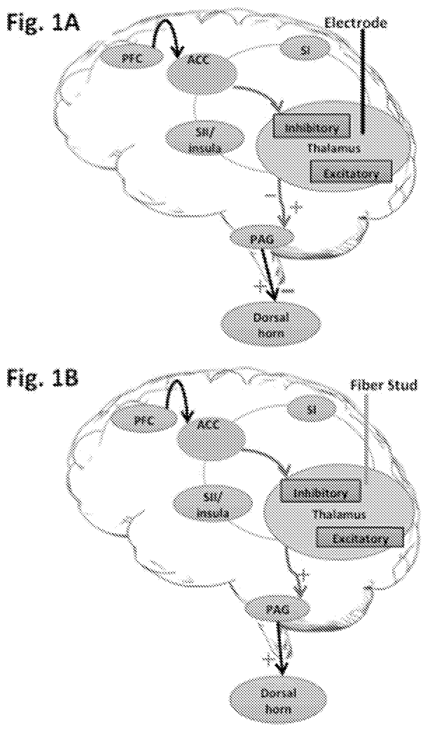

The thalamic neurons project to the dorsal hom of spinal cord via Periaqueductal gray (PAG) and modulates ascending nociceptive information. Changes in the gene expression, biochemistry, thalamic blood flow and responsiveness of thalamic neurons have been shown in the animal models for pain, which suggest role of the thalamus in modulating pain. Recent work has concentrated in testing the effect of electrical stimulation (FIG. 1A) of the Thalamus, as a strategy for pain control. However, electrical pulses delivered via electrodes implanted in the deep brain (1, 2) lack the specificity (i.e., inhibitory and excitatory) in stimulating particular group of neurons, and thus the precise involvement of specific Thalamic neurons in pain modulation remains to be determined.

The advent (3, 4) of neuronal stimulation using optogenetics has enabled highly selective activation of specific neurons with millisecond-temporal precision. In contrast to electrical stimulation, optogenetics is more specific (by genetic targeting) and multiple types of neurons can be targeted at the same region of the nervous device (5, 6). Optogenetic stimulation provides high temporal precision (4, 15, 25-28) by introducing light-activatable molecular channels such as channelrhodopsin-2 (ChR2) into cells by genetic targeting. In addition to higher temporal resolution, optogenetics has several advantages over electrical stimulation such as cellular specificity and minimal invasiveness (29). Light-induced activation of ChR2, a non-selective cation channel, results in depolarization of only those cells that express ChR2. Selective activation of neurons by ms-pulsed blue light has been demonstrated in culture (27), brain slices, as well as in small animals (30-33). This optogenetic activation method is very promising for controlling cellular activities in-vitro as well as in-vivo as it only requires light of moderate intensity (˜1 mW/mm²) that can be delivered from a light emitting diode (LED) (34). Thus, optogenetics is emerging as a valuable experimental tool and a promising approach for intervening variety of neurological disorders such as blindness (7-9), drug-addiction (11, 12), and conditioned fear (13) in animal models.

Many forms of chronic pain are still resistant to conventional clinical approaches that have been targeting peripheral and spinal dorsal horn. Further, use of pharmaceutical drugs (agonists and antagonists) is not highly specific for targeted population. For example, bicuculine has major disadvantages: it is a competitive inhibitor (i.e., its efficacy will depend on the local GABAergic tone of the area) and unstable. To address this, promoter-driven expression of opsin (e.g. ChR2) was achieved in specific types of neurons (e.g. excitatory pyramidal neurons, FIG. 1B) in a targeted region of Thalamus (e.g., VAL, VPL, VM, VPMpc). The localized delivery of Opsin can be achieved by Adeno Associated or lenti virus injection. The present invention provides method and device for optical stimulation of targeted region(s) of the Thalamus in order to alleviate pain.

One of the examples where Opsin has been used in the past for pain modulation is by stimulation of excitatory neurons of the Anterior Cingulate Cortex (ACC). Optogenetic stimulation of inhibitory neurons in ACC led to decreased electrical activity in ACC, and significant reduction in pain response due to skin irritation and noxious stimulation in animals (35).

The disclosed invention includes methods for optically modulating pain in animals and human. The invention also provides device(s) for controlled modulation neural and cellular activities in brain, eye and peripheral nervous systems in order to treat different forms of chronic pain.

In one aspect, the disclosed invention provides method for the use of opsin for modulating pain, wherein optical stimulation of specific neurons in targeted regions of the Thalamus sensitized by opsin, using genetic technologies, leads to significant reduction of pain perception to noxious stimuli. Advantages of this invented approach is the fact that it targets specific neurons in targeted regions of the Thalamus, unlike global stimulation by existing electrical/magnetic approaches; thus generating better efficacy for modulating pain.

The present inventor investigated if optogenetic stimulation of excitatory neurons in different regions (VAL, VPL, VM, VPMpc) of Thalamus can modulate pain in animal model. The results presented herein show efficient pain inhibition in awakened mice subjected to optogenetic stimulation of ChR2-sensitized excitatory neurons in Thalamus. The results also demonstrated that the pain modulation is dependent on the frequency and intensity of optogenetic stimulation.

In another aspect, the present inventor demonstrated that use of different Bioluminescent Bandwidth engineered Opsins (B2EO-1, 2) leads to passive modulation of the cells by their activity-induced emission of bioluminescence, which when applied to neurons, fibroblasts, astrocytes/glia, immune cells, keratinocytes and/or vascular endothelial cells of central/peripheral nervous system leads to down-regulation of release of neurotransmitters, or release of pro-inflammatory cytokine(s); or up-regulation of endorphins and anti-inflammatory agents leading to pain inhibition.

For example, B2EO-1 when delivered to inhibitory pain neurons by use of promoters such as GAD65, SST, or NPY, upon light illumination, the release of GABA from inhibitory neurons will be enhanced, which will silence activities of pyramidal neurons involved in pain. Similarly, the vascular endothelial cells in central/peripheral nervous system, can be sensitized with the B2EO-1 (SEQ ID NO: 2) by use of promoters including but not limited to human VWF, or Tie1. The release of endorphins and anti-inflammatory agents such as opioid peptide can be enhanced upon light illumination, thus reducing pain.

B2EO-2 when expressed in excitatory neurons of targeted nervous system regions by use of promoter such as CaMKIIa, Thy1, or human synapsin 1, it will down regulate release of neurotransmitters ATP, Glutamate, and/or BONF upon light illumination. Similarly, upon expression of B2EO-2 in gial cells, their activities can be silenced by light and thus, pain. The dural fibroblasts in brain are known to release pro-inflammatory cytokine(s) such as IL-6 that stimulate dural afferents and enhance hyper-excitability leading to headache. Therefore, these fibroblasts can be specifically targeted by use of promoters, human MoMLV, or Col1α1; and upon sensitization with the B2EO-2 (SEQ ID NO: 3) light controlled suppression of release of pro-inflammatory cytokine(s) can be achieved, thus reducing migraine pain.

Dorsal root Ganglion (DRG) neurons of peripheral nervous system relay nerve injury-related primary afferent input to the spinal cord. The soma of DRG neurons are surrounded by small satellite glial cells (SGCs), which are connected by gap junctions and support DRG neurons by supplying nutrients and buffering extracellular ion and neurotransmitter levels. The interaction of SGCs and neurons vía paracrine signaling promote the pain-sensitization of peripheral nerves leading to chronic pain. The astrocytes, glia including SGCs in central/peripheral nervous system can be selectively sensitized with the B2EO-2 (SEQ ID NO: 3) by use of promoters such as GFAP, MBP,CMV, or U1snRNA; and controlled by light so as to down-regulate the release of neurotransmitters and ATP, thus reducing hyperexcitability of neurons toward pain.

immediately after nerve injury in diabetes or spinal stenosis, and other events leading to neuropathic pain, the resident immune cells, mast cells and macrophages are known to be activated which release pro-inflammatory cytokines, and chemokines. The immune cells, including macrophages and/or mast cells in central/peripheral nervous system, when specifically sensitized with the B2EO-2 (SEQ ID NO: 3) using promoters such as c-kit, ST2, or IL1RL1, their activities and thus release of pro-inflammatory cytokines can be controlled by light activation of B2EO-2. This leads to reduction of the release of histamine and pro-inflammatory reagents, thus reducing pain.

In another embodiment, the present invention includes methods and uses of the B2EO-1, or B2EO-2 for treatment of pain: wherein the use comprises delivery of the B2EO-genes to different cells of different organs by either chemical, viral or physical transduction method; wherein activation of B2EO is achieved upon intrinsic bioluminescence (in presence of co-factor such as furimazine or its analogs); and wherein an effect is measured by an electrophysiology or other functional and behavioral analysis.

According to yet another aspect of the invention, method of efficient pain inhibition uses eyes as optical windows to the pain perception circuits of the brain. In this method, suitable wavelength and mode of light stimulation of eye led to significant inhibition of pain in awakened animals.

In another aspect, the invention provides device(s) for controlled modulation of neural and other cellular activities in brain, eye and peripheral nervous systems in order to treat different forms of chronic pain.

According to another aspect of the invention, the invention includes a method for inhibition of pain without use of exogenous opsin, wherein visual stimulation of eye (having endogenous opsin) is carried out using specific wavelength and frequency of visible light. In this method, suitable wavelength and mode of light stimulation of eye led to significant inhibition of pain in awakened animals. The advantage of this aspect of the invention is the use of eyes as optical window to the pain perception circuits of the brain and use suitable wavelength(s) and mode of light stimulation of eye for pain modulation. Further, visual stimulation was observed to modulate sleep patterns in animals.

In another aspect, the invention provides device(s) for controlled modulation neural activities in brain and eye in order to treat different forms of chronic pain.

Below, the presently disclosed invention will be further described by way of examples, which are provided for illustrative purposes only and accordingly are not to be construed as limiting the scope of the invention.

### EXAMPLES

Example 1—FIG. 1A illustrates Schematics of pain descending pathway during electrical versus optogenetic stimulation of nervous device. Electrical stimulation used in deep brain stimulation utilizes metal electrode (FIG. 1A) is not specific in stimulating different types of neurons and thus both inhibitory and excitatory neurons are stimulated. With optogenetic stimulation using light delivered by waveguide such as optical fiber, only cell-specific (inhibitory/excitatory) neurons are modulated in targeted Thalamic and other brain regions at tip of the fiber (FIG. 1B). This led to controlled modulation of pain by selective enhancement (+) of the neural circuitries (e.g Thalamus-PAG-Dorsal horn).

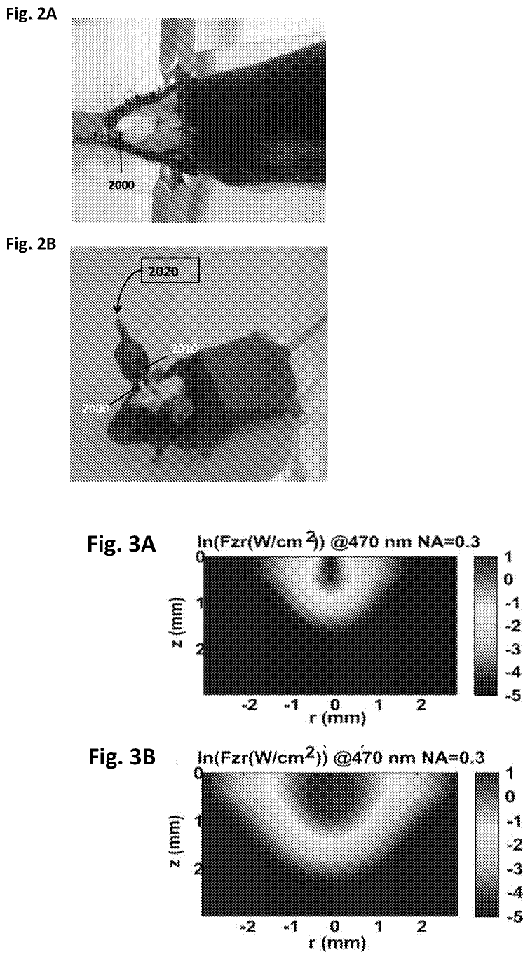

Example 2—FIG. 2A and FIG. 2B show optical neural stimulator used for stimulating thalamic region in mice. The mice (expressing ChR2 in Thalamus) were anesthetized with Ketamine (90 mg/Kg)/Xylaxine (10 mg/Kg). The mice were mounted using Stereotaxic frame, with hair removed from the head. A micro-drill was used to make three holes of size ˜300 μm on the skull, one on top of the thalamus and two for mounting screws. As shown in FIG. 2A, optical fiber stubs (diameter: 200 μm) were stereotaxically inserted and fixed using dental cement. A fiber coupled to LED (emission wavelength: 465 nm) was aligned to couple to the fiber stub for the delivery of light. The stimulating light source (LED) is connected external power supply via lead wires (FIG. 2B) so as to deliver light of controlled intensity, pulse width and frequency.

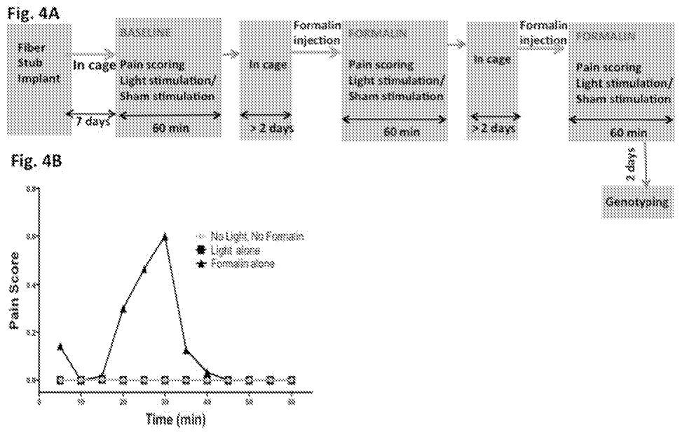

Example 3—FIG. 3A and FIG. 3B show results of simulation for choice of laser power in order to stimulate specific regions of the Thalamus. In order to determine the parameters for light delivery (e.g. intensity) to the Thalamus in a controlled manner, Monte Carlo (MC) simulation software (BeamMCML) was developed based on the widely used MCML software (36), which is capable of simulating light in multi-layered media. First, collimated point light was considered, and then the convolution method (37) was used to obtain the simulated 470 nm light beam propagation. Briefly, by use of a random number evenly distributed between 0 and 1, the relationship was determined among the random number and a launch radius following a Gaussian probability density function with 1/e intensity radius. To account for the diverging source, defined by the numerical aperture (NA), the azimuthal angle is determined by a random number uniformly distributed from 0 to 2π and the elevation is determined from another random number distributed uniformly from 0 to sin−1(NA) (38). Monte Carlo simulation of 470 nm light propagation in brain, delivered by optical fiber with NA=0.3 was evaluated using tissue-optical properties (μₐ=Absorption coefficient; μₛ=Scattering coefficient; g=anisotropy factor; n=refractive index) of the mouse Grey matter as 3.7 cm⁻¹, 110 cm⁻¹, 0.89 and 1.37; and that of white matter as 3.5 cm⁻¹, 430 cm⁻¹, 0.8 and 1.37 (39). The photon fluence distribution inside brain were extracted from the simulation results and graphed for comparisons between different incident intensities. As shown, both the penetration depth and lateral spread of the 470 nm light increased upon increasing the incident light power from 5 mW (FIG. 3A) to 50 mW (FIG. 3B).

Example 4—Pain assay using skin-irritant (Formalin) was carried out to evaluate the effect of light stimulation of thalamus. The formalin test is a widely used tonic model of continuous pain involving neurogenic, inflammatory, and central mechanisms of nociception. Both transgenic and wild type mice were randomly assigned to receive either sham stimulation or laser stimulation. Mice generated from breeding of Thy1-ChR2 male mouse with two female wild type mice was used for the assay. Behavioral testing was performed by an observer unaware of the expression of ChR2 in the mice. Each mouse was randomly assigned to receive light stimulation or sham stimulation. Therefore the mice were allocated to one of two groups (1: mice without light treatment; 2: mice with light stimulation). Approximately 7 days after implanting the fiber stub, the fiber coupled to LED (emission wavelength: 465 nm) was aligned to couple to the fiber stub. A pre-formalin baseline test was performed in which all mice received sham stimulation (no light) for 60 min. The time the mice spent on lifting and licking the paw was calculated and the pain sensation was scored as follows: paw lifting as 1 and paw licking as 2. Then, another baseline (with light alone, but no formalin) pain scoring was carried out. After these two pre-formalin baselines, the fiber coupled to LED was removed and mice returned to their cages.

After more than 2 days in the cages, the mice were subjected to the formalin test (40) where each mouse tested individually in an observation chamber for the 60 min test period. As with the pre-formalin test, the fiber coupled to LED was aligned to couple to the fiber stub. Animals were then administered a 20 μL subcutaneous injection of 1% formalin into the dorsal hind paw of the mouse using a micro-syringe with a 26-gauge needle. The light stimulation (or sham stimulation in interval of more than 2 days) was then applied and the amount of time the animal spent lifting or licking the injected paw was recorded during the 60 minute test period. The power of light delivered from the fiber was set at 10 mW. The light flash continued with repetition rate of 10 Hz and pulse width of 10 ms (i.e., 10% duty cycle during each stimulation cycle). FIG. 4A illustrates the experimental scheme for evaluating modulation of Formalin induced pain by optical stimulation of Thalamus. At the end of all experiments, the mice was sacrificed and tissue harvested for genotyping to check expression of ChR2. FIG. 4B shows pre-formalin baseline (No Light, No Formalin and Light alone) pain score as a function of observation time. No detectable pain was observed in mice (with or without ChR2 expression in thalamus) during these pre-formalin baseline measurements. However, after formalin injection (in absence of stimulation light) the pain score of same mouse increased significantly over the observation period (60 min) as shown in FIG. 4B. The pain scores in all pre-formalin groups were found to be lower than 0.2 (FIG. 4B), and no significant difference was observed among the groups without and with light stimulation. This confirms that the light stimulation does not modulate behavior in the absence of noxious pain stimuli.

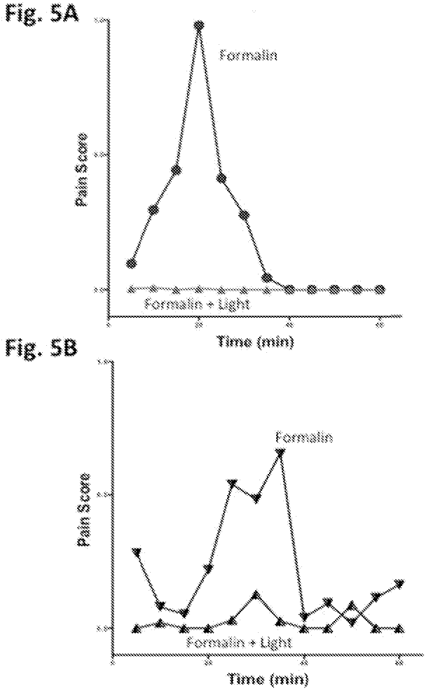

Example 5—To evaluate whether or not optogenetic stimulation of neurons in the Thalamus would modulate pain behavior, the formalin test assay of acute inflammatory pain (40) was conducted 1 wk after implantation of the fiber stub. FIG. 5A and FIG. 5B show two examples of Behavioral assay of pain modulation by optogenetic stimulation of different thalamic regions. Based on the MC simulation (FIG. 3), the known threshold for ChR2-stimulation, the power of the incident light was selected for in-vivo behavioral modulation study. At a depth of 0.8 mm (extent of Thalamus), the stimulating blue laser beam gets attenuated by 90%. Therefore, incident laser intensity of ˜30 mW/mm² was used for behavioral studies so that at depth of 0.8 mm (size of Thalamic regions such as VPL, VPM) from the fiber tip, the effective light intensity is ˜3 mW/mm², which is around threshold intensity required for optogenetic stimulation. This illumination intensity corresponds to an activation volume of ˜0.1 mm³ (size of Thalamic regions such as VPL, VPM). FIG. 5A shows pain scores as a function of time after formalin injection for Mouse-1 in which Ventral anterior-lateral complex (VAL), ventral posterolateral nucleus (VPL) and ventral medial nucleus (VM) of thalamus was targeted for light stimulation. The pain scoring as a function of time after formalin injection for Mouse-2 (with Ventral medial nucleus and parvicellular part of ventral posteromedial nucleus of thalamus being targeted) is shown in FIG. 5B.

As expected, the pain scores following the formalin injection (FIG. 5A and FIG. 5B) were higher than the scores in pre formalin test (FIG. 4B). Of primary interest is that formalin induced less pain in ChR2-expressing mice with light stimulation of thalamic regions. Switching off the light stimulation led to increased pain sensation in these mice (FIG. 5A and FIG. 5B). It is important to note that the laser stimulation of excitatory neurons of Thalamic regions expressing ChR2 in freely moving mice caused a dramatic and significant inhibition of pain (FIG. 5A and FIG. 5B). Further, the neural activities was found to decrease on the side of the thalamus receiving input from the chronically painful site. Optogenetic stimulation of thalamic regions (receiving input from the chronically painful site) reduced the pain sensation.

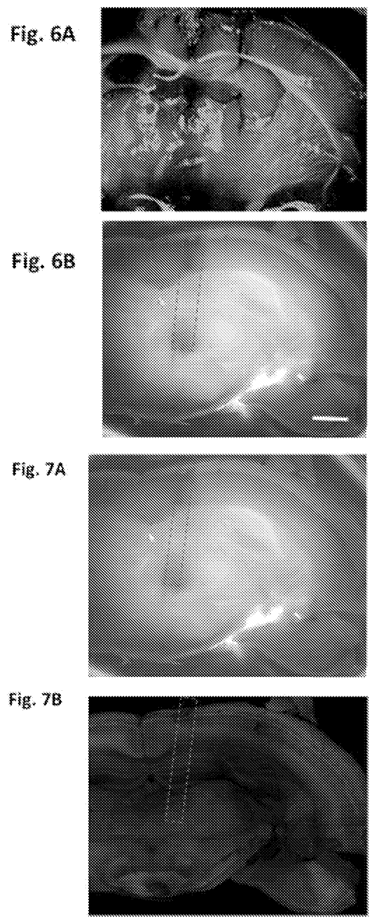

Example 6—Following behavioral measurements, mice were sacrificed and brains were removed carefully and post-fixed in 4% PFA for 24 to 48 hrs, and subsequently transferred to PBS. The brain tissues were sliced at 500 μm sections using Bain slicer (Zivic Instruments). Slices were then mounted on slides and the location of the fiber stub in each slice was examined “blind” to behavioral outcome and group designation for all mice using a upright laser scanning confocal microscope (Olympus Fluoview 1000) with a 2.5× objective. FIG. 6A illustrates confocal laser scanning reflectance microscopy of coronal section of Mice 1 showing stimulation of Ventral anterior-lateral complex (VAL), ventral posterolateral nucleus (VPL) and ventral medial nucleus (VM) of thalamus. The location of optical fiber is shown by dotted rectangle. In FIG. 6B, the confocal laser scanning transmittance microscopy of Coronal section of Mice 2 is shown, where stimulation of Ventral medial nucleus (VM) and parvicellular part of ventral posteromedial nucleus (VPMpc) of thalamus is carried out.

Optogenetic stimulation of specific Thalamic and other deep brain regions created tremor and paralysis of awakened animals. The strength of tremor and paralysis was dependent on intensity and frequency of optogenetic stimulation of brain. Further, optogenetic stimulation of specific Thalamic and other deep brain regions created roll and yaw motion of the head.

Example 7—Here, histological (FIG. 7A) and fluorescence (FIG. 7B) imaging of ChR2-YFP expression at the distal end of the implanted fiber stub in the mouse brain is shown. Correlation of confocal fluorescence (FIG. 7B) with transmission image (FIG. 7A) of the coronal section of the mouse brain shows reporter-YFP Fluorescence in targeted regions (VM, VPMpc) of thalamus near the distal tip of the inserted fiber stub in Mice 2 (FIG. 7B). Mice showing no YFP expression (and confirmed after genotyping) were considered as wild type. The light stimulation did not modulate pain score in wild type mice. Thus, the invention provides a method of chronic pain management, comprising: (i) administering to a subject's brain adeno-associated/lenti virus which delivers any of the opsin-encoding genes to targeted neurons, but preferably the opsins that are activated by red-shifted wavelengths to minimize damage to brain (if any); (ii) cell-specific expression driven by for example use of promoters such as Thy1 and CamKIIa; and (iii) implantation of optical stimulator (e.g. LED source coupled to fiber). The light delivery in optical stimulator is controlled by either wired battery powered supply or via wireless transmitter-receiver.

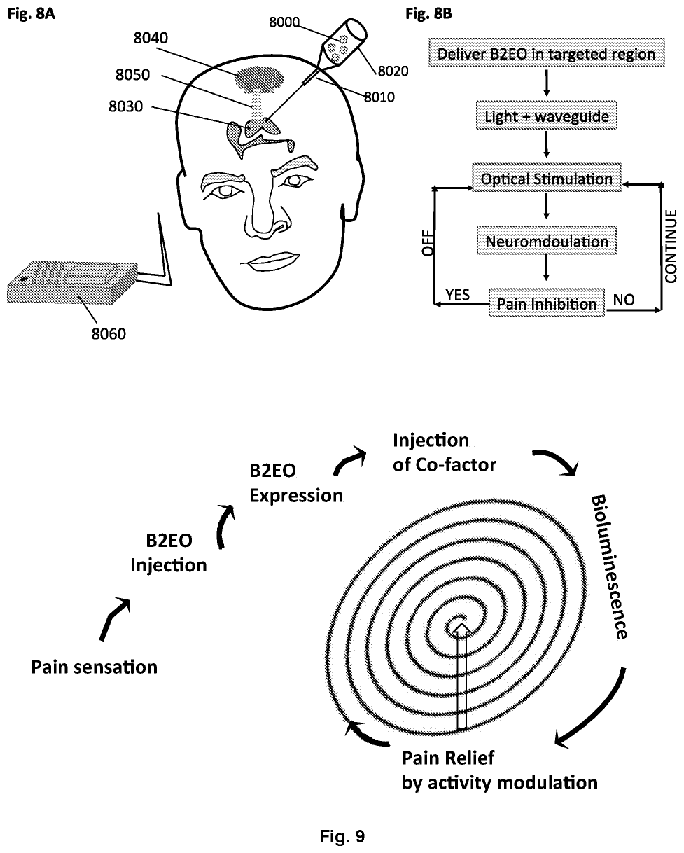

Example 8—In order to translate optogenetic stimulation for pain inhibition, an implantable optical neural stimulator device is proposed which comprises of an implantable light source and/or waveguide carrying stimulation light designed to be permanently inserted in to targeted region(s) so as to deliver light to the targeted nervous system region(s) that has been genetically modified to express an opsin. FIG. 8A shows the schematic design of delivery of light sensitizing agents (8000: B2EO) via Cannula (8010) that can be sealed using Delivery device (8020); and active optical modulation of cellular activities in targeted CNS/PNS region (8030) by the implantable LEDs (8040) and waveguides (8050). The implantable waveguide(s) is (are) made of glass, polymer, PMMA, silicone or PDMS; and is (are) coated with a biocompatible material to minimize neuro-inflammatory response without compromising the light guidance. The LED(s) is(are) controlled to generate pulses with a pulse width between 1 to 100 milliseconds, using a duty cycle between 1 to 100 percent The LED(s) and/or tip of the waveguides (8050) are arranged so as to illuminate the target area(s), wherein the tip of the waveguide(s) is (are) shaped flat or tapered so as to control the shape of emanating light beam(s). The intensity of light emanating from the light source or waveguide coupled to the light source range between 1 mW/mm² to 100 mW/mm².

An implantable power supply (in the skull, below the skin), drives the implantable light source to generate pulses of light when triggered by the patient or health care provider until switched off manually or by a pre-set program. The implantable power supply is coupled to one or more implantable inductive coils configured to receive magnetic flux from a transcutaneous magnetic flux source configured to recharge the implantable power supply The controller coupled to the light source, is also implanted and configured to result in controlled light intensity sufficient to elicit or inhibit activities in specific neurons or other cell types pre-sensitized with an opsin in targeted region(s) of the nervous system(s). The external controller (8060) is configured to wirelessly communicate with the implantable unit(s). The controller also communicates with an implantable photo-sensor that can measure intensity of delivered light inside the tissue and thus determine undesired loss that may occur during transmission from light source(s) via the waveguide(s). The implantable photo-sensor can be either a photodiode, a photovoltaic cell, a pyroelectric sensor, a photoresistor, a phototransistor, or a photoconductor; and is positioned with respect to the light-delivering waveguide (8050) so as to get a small fraction of incident light. The controller (8060) is designed to act in response to an undesired light loss level above a preset threshold by flagging the event in a software log file, and in case of an undesired light loss level above a preset threshold, the controller is programmed to stop the power supplied to the light source(s). Furthermore, cellular activity sensor (impedance sensor, a capacitance sensor, an electroneurogram sensor, or an electroencephalogram sensor) is placed near the targeted tissue to measure the light-activated cellular signal from the targeted nervous system region(s). The controller (8060) is designed to act in response to a preset out-of-range signal detected by the cellular activity sensor by stopping the power supplied to the light source (8040).

FIG. 8B shows the flow chart for optical modulation of cellular activities in CNS/PNS for pain inhibition. For example, B2EO-2 when expressed in excitatory neurons of targeted nervous system regions by use of promoter such as CaMKIIa, Thy1, or human synapsin 1, it down-regulates release of neurotransmitters ATP, Glutamate, and/or BDNF upon light illumination. Similarly, upon expression of B2EO-2 in gial cells, their activities are silenced by light and thus, pain.

Example 9—FIG. 9 shows the mechanism for B2EO based passive modulation of cellular activities in CNS/PNS for pain inhibition. Once chronic pain sensation persists, B2EO under control of a promoter, is packaged in an adenovirus, adeno-associated virus or lentivirus vector, and is injected to the target organ(s) to allow expression in targeted cells. 2-4 weeks after injection, B2EO expression in specific cells of target organ occurs. Then, the co-factor (Furimazine or its analog) is injected intravenously or orally taken to be delivered to the desired CNS/PNS region(s). The targeted, already-activated, hyper sensitized pain neurons or other (fibroblast/glia, immune or endothelial) cells expressing B2EO in presence of co-factor generates bioluminescence light. This intrinsic bioluminescence light up-regulates the activities of the targeted cells expressing B2EO-1 and down-regulates activities of targeted B2EO-2 expressing cells in order to attenuate pain sensation. Re-injection or uptake of co-factor is carried out at intervals to mitigate recurrence of pain sensation.

Dorsal root Ganglion (DRG) neurons of peripheral nervous system relay nerve injury-related primary afferent input to the spinal cord. The soma of DRG neurons are surrounded by small satellite glial cells (SGCs), which are connected by gap junctions and support DRG neurons by supplying nutrients and buffering extracellular ion and neurotransmitter levels. The interaction of SGCs and neurons via paracrine signaling promote the pain-sensitization of peripheral nerves leading to chronic pain. The astrocytes, glia including SGCs in central/peripheral nervous system can be selectively sensitized with the B2EO-2 (SEQ ID: 3) by use of promoters such as GFAP, MBP,CMV, or U1snRNA: and controlled by self-generated bioluminescent light so as to down-regulate the release of neurotransmitters and ATP, thus reducing hyperexcitability of neurons toward pain. Similarly, the dural fibroblasts in brain are known to release pro-inflammatory cytokine(s) such as IL-6 that stimulate dural afferents and enhance hyper-excitability leading to headache. Therefore, these fibroblasts are specifically targeted by use of promoters, human MoMLV, or Col1α1; and upon sensitization with the B2EO-2 (SEQ ID: 3), self-generated bioluminescent light controlled suppression of release of pro-inflammatory cytokine(s) is achieved, thus reducing migraine pain.

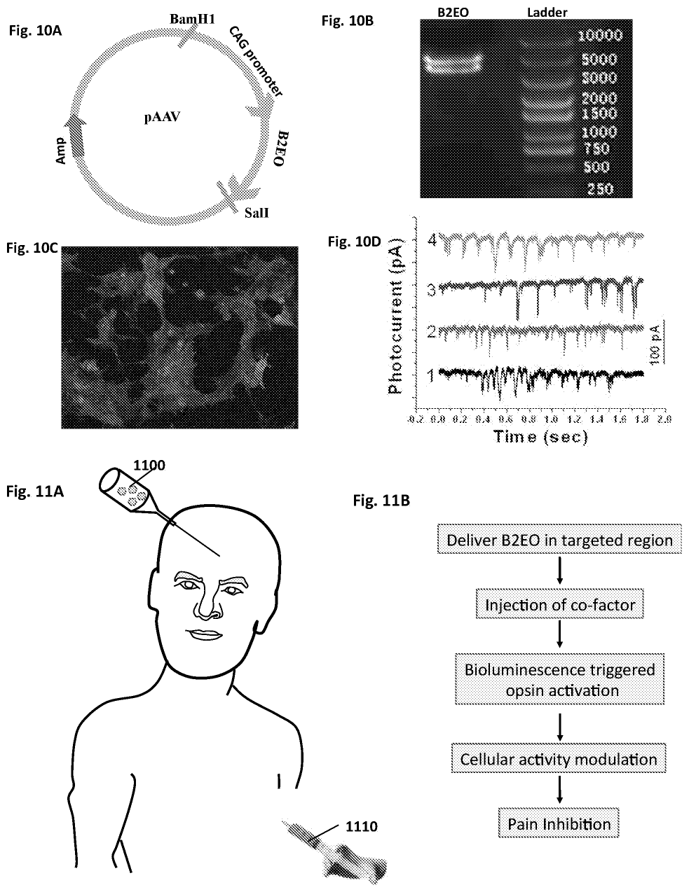

Example 10—FIG. 10A shows the map of plasmid encoding Bioluminescent Bandwidth engineered Opsin (B2EO). FIG. 10B shows the gel electrophoresis of B2EO after restriction digest using restriction enzymes, BamH/SalI. Delivery of the B2EO-genes to different cells of different organs can be achieved in a promoter-specific manner by either chemical, viral or physical transduction method; wherein activation of B2EO is achieved upon intrinsic bioluminescence (in presence of co-factor such as furimazine or its analogs); and wherein an effect is measured by an electrophysiology or other functional and behavioral analysis. FIG. 10C shows the fluorescence image of B2EO-1 expressing HEK293 cells 2 days after lipofection. FIG. 10D shows the representative inward current in B2EO-1 expressing cells in response to a light (average intensity: 0.09 mW/mm²) pulse (in trace 1) measured by Patch-clamp electrophysiology. Recurrence of inward photocurrent is evident in traces 2, 3 & 4 even after switching off the initial light pulse (during trace 1).

B2EO-1 when delivered to inhibitory pain neurons by use of promoters such as GAD65, SST, or NPY, upon intrinsic bioluminescence generation, the release of GABA from inhibitory neurons is enhanced, which silences the activities of pyramidal neurons involved in pain. Similarly, the vascular endothelial cells in central/peripheral nervous system, can be sensitized with the B2EO-1 (SEQ ID: 2) by use of promoters including but not limited to human VWF, or Tie1. The release of endorphins and anti-inflammatory agents such as opioid peptide is enhanced upon bioluminescence light generation, thus reducing pain.

Example 11—FIG. 11A shows the B2EO based passive modulation of cellular activities in CNS/PNS for pain inhibition. B2EO nucleotide comprises of a sequence comprising at least 75%, 85%, 95% or 100% identity to SEQ ID NO: 2, or 3 wherein the encoded polypeptide is activated by intrinsic bioluminescence in presence of an injected co-factor. A vector comprising the nucleic acid encoding for B2EO, is selected from an adenovirus, adeno-associated virus or lentivirus vector The delivery of Bioluminescent Bandwidth engineered Opsin (B2EO) under control of a promoter, is carried out by injection (1100) into targeted tissue or via intravenous injection of the virus vector carrying B2EO. 2-4 weeks after injection, B2EO expression in specific cells of target organ occurs. Then, the co-factor (Furimazine or its analog) is injected intravenously (110) or orally taken to be delivered to the desired CNS/PNS region(s). FIG. 11B shows the flow chart for B2EO based passive modulation of cellular activities in CNS/PNS for pain inhibition, wherein activation of B2EO is achieved upon intrinsic bioluminescence (in presence of co-factor such as furimazine or its analog). Once the co-factor (furimazine or its analog) is depleted, and if the pain sensation still persists, the co-factor is re-injected or orally administered at intervals suitable for treating pain.

Immediately after nerve injury in diabetes or spinal stenosis, and other events leading to neuropathic pain, the resident immune cells, mast cells and macrophages are known to be activated which release pro-inflammatory cytokines, and chemokines. The immune cells, including macrophages and/or mast cells in central/peripheral nervous system, when specifically sensitized with the B2EO-2 (SEQ ID: 3) using promoters such as c-kit, ST2, or IL1RL1, their activities and thus release of pro-inflammatory cytokines is controlled by bioluminescence light activation of B2EO-2. This leads to reduction of the release of histamine and pro-inflammatory reagents, thus reducing pain.

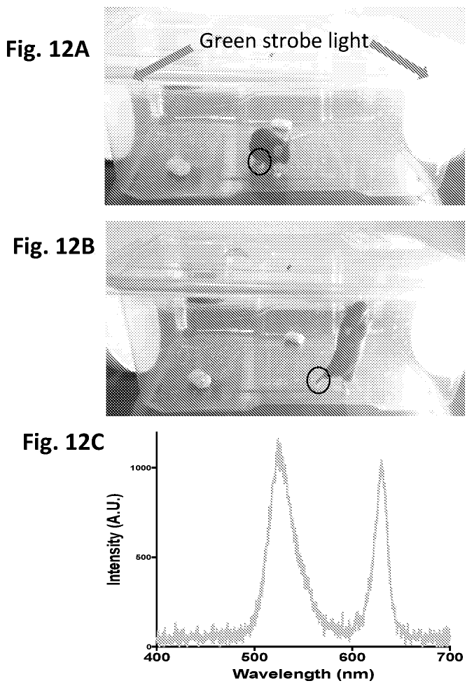

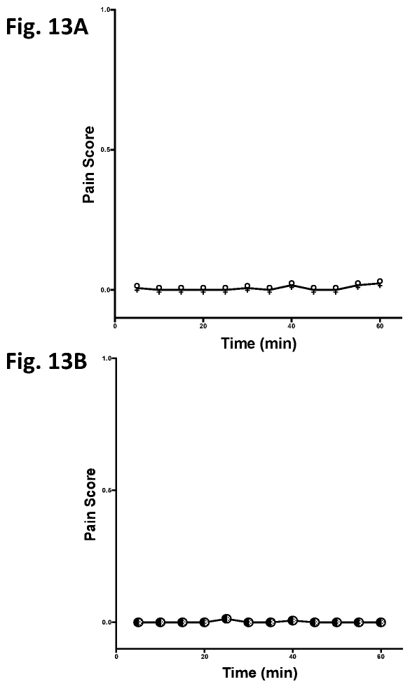

Example 12—In order to evaluate the whether pain can be inhibited by visual stimulation (using eyes as optical windows to the pain perception circuits of the brain), different wavelength bands and mode of light stimulation (e.g. frequency) was examined. The mice were individually placed inside optically-transparent cages (with water and food) between two counter-propagating light beams. FIG. 12A shows a set up for optical stimulation of eye to evaluate effect on pain modulation. The counter-propagating strobe lights was applied just after injecting 1% formalin (20 micro liters) to right hind paw of a mouse. Though swelling and redness observed in the injected paw (encircled), the mice were seen to walk naturally inside the cage. At approximately 40 min after injecting formalin to right hind paw of the mouse, it was observed to be standing using two hind paws and explore the environment (FIG. 12B). Strobe visible light in the range of 1-2 Hz was used for visual stimulation, which is expected to generate slowest and high amplitude delta waves in the brain. Though these waves are so far believed to be generated in deep, dreamless sleep (stage-4), the visual stimulation at 1-2 Hz are found to generate these waves in fully-awake behaving animals. The slow delta waves in the brain were found to inhibit pain perception.

In the present invention, use of blue light (400-500 nm) was avoided to minimize damage to retina via heating and photo-chemical toxicity. Further, the band 550-600 nm was excluded to minimize absorption by blood (and thus avoid any potential damage to eye). The spectrum of the optimized strobe light used for modulating pain is shown in FIG. 12C.

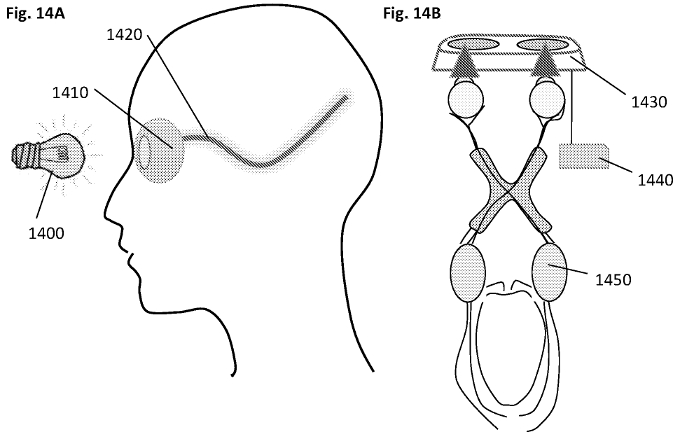

Example 13—To evaluate whether or not optical stimulation of photoreceptors in the retina would modulate pain behavior, the formalin test assay of acute inflammatory pain (40) was conducted. FIG. 13A and FIG. 13B shows results of behavioral assay of pain modulation by optical stimulation (˜0.04 mW/mm²) of eyes of wild-type mice The formalin induced less pain in visually-stimulated mice (FIG. 13A and FIG. 13B) than the non-stimulated mice (FIG. 4B). It is important to note that the visual stimulation of retina in freely moving mice caused a dramatic and significant inhibition of pain (FIG. 13A and FIG. 13B). Optical stimulation of eyes using optical retinal stimulator enhances the release of endorphins and anti-inflammatory agents such as opioid peptide, thus reducing pain.

Example 14—FIG. 14A shows the macro level depiction of active optical stimulation of eye for pain inhibition. The subject with normal functioning retina (1410) and non-injured optic nerve (1420) is illuminated with strobe light (1400), whose peak and width of the spectrum can be tuned from blue (400 nm) to red (700 nm) wavelength using choice of LEDs and/or band pass filters. A controller for light source (1400) modulates the light intensity in the range of 0.01 to 10 candela. Further, the controller and application software is used for varying the frequency of strobing in the range of 0.1 to 5 Hz. FIG. 14B shows the detailed depiction of circuitry for optically-stimulated eye induced modulated brain activities for pain inhibition. The Light projecting goggles (1430) is battery powered and wirelessly controlled or connected with power-supply and controller (1440). In case of application of light during closed-eye condition (or sleep), the optical retinal stimulator (1430) will target the intrinsically-photosensitive RGCs. The wearable battery-powered/controlled device comprises of array of Light Emitting Diodes (LEDs) emitting between red (600 nm) to near infrared (800 nm) wavelength range, and with light intensity in the range of 0.01 to 10 candela, illuminating the eyes with the eyelids closed at a rate of 0.1 to 5 Hz for 1 to 100 min.

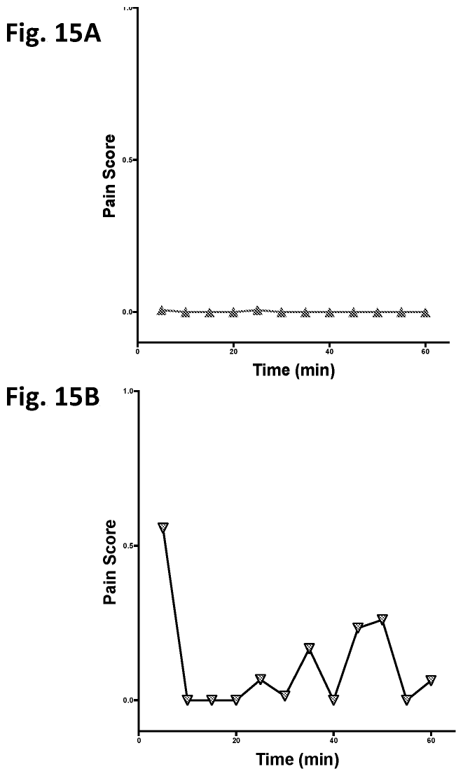

Example 15—1410: in absence of intact photosensitivity of retina (in case of no light perception due to retinal degenerative diseases), the optical retinal stimulator may not realize the goal of pain inhibition. In order to evaluate the effect of optical retinal stimulator in inhibiting pain of photo-sensitized retina, mice with ChR2-opsin expression in retinal ganglion cells are used. FIG. 15A shows the behavioral assay of pain modulation by optical stimulation of Thy1-ChR2 sensitized eye. The pain score was obtained as weighted average of lifting (weight=1), and licking (weight=2) of injected paw. Pain scoring as a function of time after formalin injection (to hid paw) of Mouse-1 with Thy1-ChR2 sensitized eye in presence of visual stimulation. FIG. 15B shows the pain scoring as a function of time after formalin injection (to hid paw) of Mouse-2 with Thy1-ChR2 sensitized eye in presence of visual stimulation. The formalin induced less pain in visually-stimulated mice with with Thy1-ChR2 sensitized eye (FIG. 15A and FIG. 15B) than the non-stimulated mice.

TABLE-01
Amino acid and DNA sequences of Bioluminescent
Bandwidth engineered Opsin -1 (B2EO-1)

Amino acid sequence:
MDYGGALSAVGRELLFVTNPVVVNGSVLVPEDQCYCAGWIESRGTNGAQTASNVLQWLAA
GFSILLLMFYAYQTWKSTCGWEEIYVCAIEMVKVILEFFFEFKNPSMLYLATGHRVQWLR
YAEWLLTCPVILIHLSNLTGLSNDYSRRTMGLLVSDIGTIVWGATSAMATGYVKVIFFCL
GLCYGANTFFHAAKAYIEGYHTVPKGRCRQVVTGMAWLFFVSWGMFPILFILGPEGFGVL
SVYGSTVGHTIIDLMSKNCWGLLGHYLRVLIHEHILIHGDIRKTTKLNIGGTEIEVETLV
EDEAEAGAMAELISSATRSLFAAGGINPWPNPYHHEDMGCGGMTPTGECFSTEVVWCDPSY
GLSDAGYGYCFVEATGGYLVVGVEKKQAWLHSRGTPGEKIGAQVCQWIAFSIAIALLTFY
GFSAWKATCGWEEVYVCCVEVLFVTLEIFKEFSSPATVYLSTGNHAYCLRYFEWLLSCPV
ILIRLSNLSGLKNDYSKRTMGLIVSCVGMIVFGMAAGLATDWLKWLLYIVSCIYGGYMYF
QAAKCYVEANHSVPKGHCRMVVKLMAYAYFASWGSYPILWAVGPEGLLKLSPYANSIGHS
ICDIIAKEFVVTFLAHHLRIKIHEHILIHGDIRKTTKMEIGGEEVEVEEFVEEEDEDTVVS
KGEEDNMVSKGEEDNMASLPATHELHIFGSINGVDFDMVGQGTGNPNDGYEELNLKSTKG
DLQFSPWILVPHIGYGFHQYLPYPDGMSPFQAAMVDGSGYQVHRTMQFEDGASLTVNYRY
TYEGSHIKGEAQVKGTGFPADGPVMTNSLTAADWCRSKKTYPNDKTIISTFKWSYTTGNG
KRYRSTARTTYTFAKPMAANYLKNQPMYVFRKTELKHSKTELNFKEWQKAFTGFEDFVGD
WRQTAGYNLDQVLEQGGVSSLFQNLGVSVTPIQRIVLSGENGLKIDIHVIIPYEGLSGDQ
MGQIEKIFKVVYPVDDHHFKVILHYGTLVIDGVTPNMIDYFGRPYEGIAVFDGKKITVTG
TLWNGNKIIDERLINPDGSLLFRVTINGVTGWRLCERILA (SEQ ID No: 1)
DNA sequence:
ATGGATTACGGTGGTGCACTTTCGGCCGTGGGGCGTGAGCTGCTATTCGTGACGAACCCG
GTAGTCGTCAACGGCTCAGTCCTTGTCCCCGAAGACCAATGCTATTGTGCAGGATGGATA
GAAAGCAGAGGCACGAACGGCGCGCAAACGGCATCTAATGTGCTACAGTGGTTAGCTGCA
GGCTTTTCGATACTTCTATTGATGTTTTATGCATACCAAACATGGAAATCTACATGCGGCTG
GGAAGAGATTTATGTCTGCGCAATCGAAATGGTGAAGGTTATCCTGGAGTTCTTCTTTGAAT
TCAAAAACCCTTCAATGTTGTATCTCGCAACAGGGCATAGAGTCCAGTGGTTAAGGTACGC
AGAGTGGCTCTTGACTTGTCCTGTTATACTTATACACCTGAGCAATCTCACTGGCTTAAGTA
ATGATTATAGCCGACGGACAATGGGCTTGTTAGTTTCAGACATCGGAACTATCGTATGGGG
GGCCACAAGCGCTATGGCCACTGGCTACGTTAAGGTAATTTTTTTTTGCCTGGGATTGTGT
TACGGCGCAAACACATTTTTCCATGCAGCAAAAGCTTATATTGAAGGTTACCATACAGTTCC
CAAAGGACGGTGCAGACAAGTCGTCACAGGAATGGCGTGGCTGTTCTTTGTAAGTTGGGG
TATGTTCCCCATATTATTCATCCTTGGCCCAGAGGGCTTTGGAGTGCTGTCTGTCTATGGAT
CTACAGTAGGTCACACTATCATTGATTTGATGAGCAAAAACTGCTGGGGCCTTCTAGGACA
CTACTTACGCGTCCTGATTCATGAACACATTCTCATTCACGGAGACATACGTAAGACGACC
AAGCTAAATATCGGAGGAACGGAAATCGAGGTTGAAACACTTGTAGAGGATGAGGCTGAA
GCTGGAGCTATGGCTGAGCTGATCAGCAGCGCCACCAGATCTCTGTTTGCCGCCGGAGG
CATCAACCCTTGGCCTAACCCCTACCACCACGAGGACATGGGCTGTGGAGGAATGACACC
TACAGGCGAGTGCTTCAGCACCGAGTGGTGGTGTGACCCTTCTTACGGACTGAGCGACGC
CGGATACGGATATTGCTTCGTGGAGGCCACAGGCGGCTACCTGGTCGTGGGAGTGGAGA
AGAAGCAGGCTTGGCTGCACAGCAGAGGCACACCAGGAGAAAAGATCGGCGCCCAGGTC
TGCCAGTGGATTGCTTTCAGCATCGCCATCGCCCTGCTGACATTCTACGGCTTCAGCGCCT
GGAAGGCCACTTGCGGTTGGGAGGAGGTCTACGTCTGTTGCGTCGAGGTGCTGTTCGTGA
CCCTGGAGATCTTCAAGGAGTTCAGCAGCCCCGCCACAGTGTACCTGTCTACCGGCAACC
ACGCCTATTGCCTGCGCTACTTCGAGTGGCTGCTGTCTTGCCCCGTGATCCTGATCAGACT
GAGCAACCTGAGCGGCCTGAAGAACGACTACAGCAAGCGGACCATGGGCCTGATCGTGT
CTTGCGTGGGAATGATCGTGTTCGGCATGGCCGCAGGACTGGCTACCGATTGGCTCAAGT
GGCTGCTGTATATCGTGTCTTGCATCTACGGCGGCTACATGTACTTCCAGGCCGCCAAGT
GCTACGTGGAAGCCAACCACAGCGTGCCTAAAGGCCATTGCCGCATGGTCGTGAAGCTGA
TGGCCTACGCTTACTTCGCCTCTTGGGGCAGCTACCCAATCCTCTGGGCAGTGGGACCAG
AAGGACTGCTGAAGCTGAGCCCTTACGCCAACAGCATCGGCCACAGCATCTGCGACATCA
TCGCCAAGGAGTTTTGGACCTTCCTGGCCCACCACCTGAGGATCAAGATCCACGAGCACA
TCCTGATCCACGGCGACATCCGGAAGACCACCAAGATGGAGATCGGAGGCGAGGAGGTG
GAAGTGGAAGAGTTCGTGGAGGAGGAGGACGAGGACACAGTG GT
GAGCAAGGGCGAGGAGGATAACATGGTGTCCAAGGGCGAAGAGGACAACATGGCCAGCC
TGCCTGCCACCCACGAGCTGCACATCTTCGGCAGCATCAACGGCGTGGACTTCGACATGG
TGGGACAGGGCACCGGCAACCCCAACGACGGCTACGAGGAACTGAACCTGAAGTCCACA
AAGGGCGACCTGCAGTTCAGCCCCTGGATTCTGGTGCCCCACATCGGCTACGGCTTCCAC
CAGTACCTGCCCTACCCCGACGGCATGAGCCCTTTCCAGGCCGCTATGGTGGATGGCAGC
GGCTACCAGGTGCACCGGACCATGCAGTTTGAGGACGGCGCCAGCCTGACCGTGAACTA
CCGGTACACATACGAGGGCAGCCACATCAAGGGCGAGGCCCAAGTGAAGGGCACAGGCT
TTCCAGCCGACGGCCCCGTGATGACCAATAGCCTGACAGCCGCCGACTGGTGCAGAAGC
AAGAAAACCTACCCCAATGACAAGACCATCATCAGCACCTTCAAGTGGTCCTACACCACCG
GCAATGGCAAGCGGTACAGAAGCACCGCCCGGACCACCTACACCTTCGCCAAACCTATGG
CCGCCAACTACCTGAAGAACCAGCCTATGTACGTGTTCCGCAAGACCGAGCTGAAGCACT
CCAAGACAGAACTGAACTTCAAAGAGTGGCAGAAAGCCTTCACCGGGTTTGAAGATTTCGT
TGGGGACTGGCGACAGACAGCCGGCTACAACCTGGACCAAGTCCTTGAACAGGGAGGTG
TGTCCAGTTTGTTTCAGAATCTCGGGGTGTCCGTAACTCCGATCCAAAGGATTGTCCTGAG
CGGTGAAAATGGGCTGAAGATCGACATCCATGTCATCATCCCGTATGAAGGTCTGAGCGG
CGACCAAATGGGCCAGATCGAAAAAATTTTTAAGGTGGTGTACCCTGTGGATGATCATCAC
TTTAAGGTGATCCTGCACTATGGCACACTGGTAATCGACGGGGTTACGCCGAACATGATCG
ACTATTTCGGACGGCCGTATGAAGGCATCGCCGTGTTCGACGGCAAAAAGATCACTGTAA
CAGGGACCCTGTGGAACGGCAACAAAATTATCGACGAGCGCCTGATCAACCCCGACGGCT
CCCTGCTGTTCCGAGTAACCATCAACGGAGTGACCGGCTGGCGGCTGTGCGAACGCATTC
TGGCGTAA (SEQ ID No: 2)

TABLE-02
DNA sequence of Bioluminescent Bandwidth
engineered Opsin -2 (B2EO-2)

DNA sequence:
ATGGAGATCGGAGGCGAGGAGGTGGAAGTGGAAGAGTTCGTGGAGGAGGA
GGACGAGGACACAGTGGCGGCGCTGCAGGAAAAAAAAAGCTGCAGCCAGC
GCATGGCGGAATTTCGCCAGTATTGCTGGAACCCGGATACCGGCCAGATG
CTGGGCCGCACCCCGGCGCGCTGGGTGTGGATTAGCCTGTATTATGCGGC
GTTTTATGTGGTGATGACCGGCCTGTTTGCGCTGTGCATTTATGTGCTGA
TGCAGACCATTGATCCGTATACCCCGGATTATCAGGATCAGCTGAAAAGC
CCGGGCGTGACCCTGCGCCCGGATGTGTATGGCGAACGCGGCCTGCAGAT
TAGCTATAACATTAGCGAAAACAGCAGCATGGACCCCATCGCTCTGCAGG
CTGGTTACGACCTGCTGGGTGACGGCAGACCTGAAACTCTGTGGCTGGGC
ATCGGCACTCTGCTGATGCTGATTGGAACCTTCTACTTTCTGGTCCGCGG
ATGGGGAGTCACCGATAAGGATGCCCGGGAATATTACGCTGTGACTATCC
TGGTGCCCGGAATCGCATCCGCCGCATATCTGTCTATGTTCTTTGGTATC
GGGCTTACTGAGGTGACCGTCGGGGGCGAAATGTTGGATATCTATTATGC
CAGGTACGCCGACTGGCTGTTTACCACCCCACTTCTGCTGCTGGACTGGC
CCTTCTCGCTAAGGTGGATCGGGTGACCATCGGCACCCTGGTGGGTGTGG
ACGCCCTGATGATCGTCACTGGCCTCATCGGAGCCTTGAGCCACACGGCC
ATAGCCAGATACAGTTGGTGGTTGTTCTCTACAATTTGCATGATAGTGGT
GCTCTATTTTCTGGCTACATCCCTGCGATCTGCTGCAAAGGAGCGGGGCC
CCGAGGTGGCATCTACCTTTAACACCCTGACAGCTCTGGTCTTGGTGCTG
TGGACCGCTTACCCTATCCTGTGGATCATAGGCACTGAGGGCGCTGGCGT
GGTGGGCCTGGGCATCGAAACTCTGCTGTTTATGGTGTTGGACGTGACTG
CCAAGGTCGGCTTTGGCTTTATCCTGTTGAGATCCCGGGCTATTCTGGGC
GACACCGAGGCACCAGAACCCAGTGCCGGTGCCGATGTCAGTGCCGCCGA
CATGGTGTCCAAGGGCGAAGAGGACAACATGGCCAGCCTGCCTGCCACCC
ACGAGCTGCACATCTTCGGCAGCATCAACGGCGTGGACTTCGACATGGTG
GGACAGGGCACCGGCAACCCCAACGACGGCTACGAGGAACGAACCTGAAG
TCCACAAAGGGCGACCTGCAGTTCAGCCCCTGGATTCTGGTGCCCCACAT
CGGCTACGGCTTCCACCAGTACCTGCCCTACCCCGACGGCATGAGCCCTT
TCCAGGCCGCTATGGTGGATGGCAGCGGCTACCAGGTGCACCGGACCATG
CAGTTTGAGGACGGCGCCAGCCTGACCGTGAACTACCGGTACACATACGA
GGGCAGCCACATCAAGGGCGAGGCCCAAGTGAAGGGCACAGGCTTTCCAG
CCGACGGCCCCGTGATGACCAATAGCCTGACAGCCGCCGACTGGTGCAGA
AGCAAGAAAACCTACCCCAATGACAAGACCATCATCAGCACCTTCAAGTG
GTCCTACACCACCGGCAATGGCAAGCGGTACAGAAGCACCGCCCGGACCA
CCTACACCTTCGCCAAACCTATGGCCGCCAACTACCTGAAGAACCAGCCT
ATGTACGTGTTCCGCAAGACCGAGCTGAAGCACTCCAAGACAGAACTGAA
CTTCAAAGAGTGGCAGAAAGCCTTCACCGGGTTTGAAGATTTCGTTGGGG
ACTGGCGACAGACAGCCGGCTACAACCTGGACCAAGTCCTTGAACAGGGA
GGTGTGTCCAGTTTGTTTCAGAATCTCGGGGTGTCCGTAACTCCGATCCA
AAGGATTGTCCTGAGCGGTGAAAATGGGCTGAAGATCGACATCCATGTCA
TCATCCCGTATGAAGGTCTGAGCGGCGACCAAATGGGCCAGATCGAAAAA
ATTTTTAAGGTGGTGTACCCTGTGGATGATCATCACTTTAAGGTGATCCT
GCACTATGGCACACTGGTAATCGACGGGGTTACGCCGAACATGATCGACT
ATTTCGGACGGCCGTATGAAGGCATCGCCGTGTTCGACGGCAAAAAGATC
ACTGTAACAGGGACCCTGTGGAACGGCAACAAAATTATCGACGAGCGCCT
GATCAACCCCGACGGCTCCCTGCTGTTCCGAGTAACCATCAACGGAGTGA
CCGGCTGGCGGCTGTGCGAACGCATTCTGGCGTAA (SEQ ID No: 3)

TABLE-03
DNA sequence of a promoter used upstream
of B2EO-sequences for targeting neurons or
other cells as an example.

GCGTTACATAACTTACGGTAAATGGCCCGCCTGGCTGACCGCCCAACGAC
CCCCGCCCATTGACGTCAATAATGACGTATGTTCCCATAGTAACGCCAAT
AGGGACTTTCCATTGACGTCAATGGGTGGAGTATTTACGGTAAACTGCCC
ACTTGGCAGTACATCAAGTGTATCATATGCCAAGTACGCCCCCTATTGAC
GTCAATGACGGTAAATGGCCCGCCTGGCATTATGCCCAGTACATGACCTT
ATGGGACTTTCCTACTTGGCAGTACATCTACGTATTAGTCATCGCTATTA
CCATGGTCGAGGTGAGCCCCACGTTCTGCTTCACTCTCCCCATCTCCCCC
CCCTCCCCACCCCCAATTTTGTATTTATTTATTTTTTAATTATTTTGTGC
AGCGATGGGGGCGGGGGGGGGGGGGGGGCGCGCGCCAGGCGGGGCGGGGC
GGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCG
GCGGCCCTATAAAAAGCGAAGCGCGCGGCGGGCG (SEQ ID No: 4)

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

Further, a molecule or method that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some references, which may include publications, patents, and patent applications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference were individually incorporated by reference.

The specification and examples herein provide a complete description of the structure and use of illustrative embodiments Although certain embodiments have been described with a certain degree of particularity, or with reference to one or more individual embodiments, those skilled in the art could make numerous alterations to the disclosed embodiments without departing from the scope of this invention. As such, the various illustrative embodiments of the devices are not intended to be limited to the particular forms disclosed. Rather, they include all modifications and alternatives falling within the scope of the claims, and embodiments other than the one shown may include some or all of the features of the depicted embodiment. For example, components may be omitted or combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

Furthermore, the claims are not intended to include, and should not be interpreted to include, means-plus- or step-plus-function limitations, unless such a limitation is explicitly recited in a given claim using the phrase(s) “means for” or “step for,” respectively.

To the extent that any specific disclosure in the references or other literature may be considered to anticipate any generic aspect of the present invention, the disclosure of the present invention should be understood to include a proviso or provisos that exclude of disclaim any such species that were previously disclosed. The aspects of the present invention, which are not anticipated by the disclosure of such literature, are also nonobvious from the disclosure of these publications, due at least in part to the unexpectedly superior results disclosed herein.

### REFERENCES

The following references, to the extent that they provide exemplary procedural or other details supplementary to those set forth above, are specifically incorporated by reference.

1-2. (canceled)

3. A method for reducing inflammatory, neuropathic pain and chronic pain in an animal or human subject comprising:
a) delivery of a Bioluminescent Bandwidth Engineered Opsin having the amino acid sequence of SEQ ID NO: 1 (B2EO-1) to the thalamus, anterior cingulate cortex (ACC) or spinal dorsal horn of the subject's brain, carried out by injection of a virus vector or by other physical/chemical methods, delivering a nucleic acid encoding for B2EO-1 under control of a glutamic acid decarboxylase 65 (GAD65) promoter, and
b) positioning an optical neural stimulator in the targeted region(s) of the subject's thalamus, ACC or spinal dorsal horn, wherein the optical neural stimulator comprises a red-light source and/or waveguide that emits red light pulses with a width from 1 to 100 milliseconds and a duty cycle from 1 to 100 percent, and tuned to a light intensity from 1 mW/mm² to 100 mW/mm², and further wherein the optical neural stimulator stimulates the cells in the thalamus, ACC or spinal dorsal horn that are transduced or transfected with a nucleic acid encoding for B2EO-1under control of a glutamic acid decarboxylase 65 (GAD65) promoter for expressing the B2EO-1 to effect optogenetic stimulation and a reduction in pain.
c) passive stimulation of specific cells in the targeted region(s) of the subject's thalamus, ACC or, spinal dorsal horn using bioluminescent light emitted by transduced endogenous cells localized in the thalamus, ACC or spinal dorsal horn that have been sensitized with B2EO-1 in presence of an injected co-factor to effect optogenetic stimulation.

4-9. (canceled)

10. The method of claim 3, wherein selection of coordinates of the targeted regions is carried out by using an imaging modality including magnetic resonance imaging, computed tomography imaging, ultrasound imaging and/or radiography.

11-26. (canceled)

26. The method of claim 3, wherein the injected co-factor is at least one selected from the group consisting of furimazine and furimazine analogs.

27. The method of claim 1, wherein the red-light source is at least one selected from the group consisting of a lamp, light emitting diode (LED), and laser.

28. The method of claim 3, wherein the optical neural stimulator isconfigured to emit light having an emission wavelength from 600 nm to 7800 nm.

29. The method of claim 3, wherein the red-light source is a lightemitting diode (LED).

30. The method of claim 3, wherein the waveguide is an optical fiber.

33. The method of claim 3, wherein the waveguide is made of glass, polymer, polymethyl methacrylate (PMMA), silicone or polydimethylsiloxane (PDMS).


SEQUENCE LISTING (4 sequences)

SEQ ID NO: 1 - protein, 1053 aa, synthetic construct
  REGION 1..1053: note = Amino acid and DNA sequences of Bioluminescent Bandwidth engineered Opsin -1 (B2EO-1)
MDYGGALSAVGRELLFVTNPVWNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILLHLSNLTGLSNDYSRRTMGLLVSDIGTIVWGATSAMATGYVKVIFFCLGLCYGANTFFHAAKAYIEGYHTVPKGRCRQWTGMAWLFFVSWGMFPILFILGPEGFGVLSVYGSTVGHTIIDLMSKNCWGLLGHYLRVLLHEHILLHGDIRKTTKLNIGGTEIEVETLVEDEAEAGAMAELISSATRSLFAAGGINPWPNPYHHEDMGCGGMTPTGECFSTEWWCDPSYGLSDAGYGYCFVEATGGYLWGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVLIRLSNLSGLKNDYSKRTMGLLVSCVGMIVFGMAAGLATDWLKWLLYIVSCIYGGYMYFQAAKCYVEANHSVPKGHCRMWKLMAYAYFASWGSYPILWAVGPEGLLKLSPYANSIGHSICDIIAKEFWTFLAHHLRIKIHEHILLHGDIRKTTKMEIGGEEVEVEEFVEEEDEDTWSKGEEDNMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTGFEDFVGDWRQTAGYNLDQVLEQGGVSSLFQNLGVSVTPIQRIVLSGENGLKIDIHVIIPYEGLSGDQMGQIEKIFKWYPVDDHHFKVILHYGTLVIDGVTPNMIDYFGRPYEGIAVFDGKKITVTGTLWNGNKIIDERLLNPDGSLLFRVTINGVTGWRLCERILA

SEQ ID NO: 2 - DNA, 3183 bp, synthetic construct
  misc_feature 1..3183: note = DNA sequences of Bioluminescent Bandwidth engineered Opsin -1 (B2EO-1)
  source 1..3183: mol_type = other DNA
atggattacggtggtgcactttcggccgtggggcgtgagctgctattcgtgacgaacccggtagtcgtcaacggctcagtccttgtccccgaagaccaatgctattgtgcaggatggatagaaagcagaggcacgaacggcgcgcaaacggcatctaatgtgctacagtggttagctgcaggcttttcgatacttctattgatgttttatgcataccaaacatggaaatctacatgcggctgggaagagatttatgtctgcgcaatcgaaatggtgaaggttatcctggagttcttctttgaattcaaaaacccttcaatgttgtatctcgcaacagggcatagagtccagtggttaaggtacgcagagtggctcttgacttgtcctgttatacttatacacctgagcaatctcactggcttaagtaatgattatagccgacggacaatgggcttgttagtttcagacatcggaactatcgtatggggggccacaagcgctatggccactggctacgttaaggtaatttttttttgcctgggattgtgttacggcgcaaacacatttttccatgcagcaaaagcttatattgaaggttaccatacagttcccaaaggacggtgcagacaagtcgtcacaggaatggcgtggctgttctttgtaagttggggtatgttccccatattattcatccttggcccagagggctttggagtgctgtctgtctatggatctacagtaggtcacactatcattgatttgatgagcaaaaactgctggggccttctaggacactacttacgcgtcctgattcatgaacacattctcattcacggagacatacgtaagacgaccaagctaaatatcggaggaacggaaatcgaggttgaaacacttgtagaggatgaggctgaagctggagctatggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggtgagcaagggcgaggaggataacatggtgtccaagggcgaagaggacaacatggccagcctgcctgccacccacgagctgcacatcttcggcagcatcaacggcgtggacttcgacatggtgggacagggcaccggcaaccccaacgacggctacgaggaactgaacctgaagtccacaaagggcgacctgcagttcagcccctggattctggtgccccacatcggctacggcttccaccagtacctgccctaccccgacggcatgagccctttccaggccgctatggtggatggcagcggctaccaggtgcaccggaccatgcagtttgaggacggcgccagcctgaccgtgaactaccggtacacatacgagggcagccacatcaagggcgaggcccaagtgaagggcacaggctttccagccgacggccccgtgatgaccaatagcctgacagccgccgactggtgcagaagcaagaaaacctaccccaatgacaagaccatcatcagcaccttcaagtggtcctacaccaccggcaatggcaagcggtacagaagcaccgcccggaccacctacaccttcgccaaacctatggccgccaactacctgaagaaccagcctatgtacgtgttccgcaagaccgagctgaagcactccaagacagaactgaacttcaaagagtggcagaaagccttcaccgggtttgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 3 - DNA, 1836 bp, synthetic construct
  misc_feature 1..1836: note = DNA sequence of Bioluminescent Bandwidth engineered Opsin -2 (B2EO-2)
  source 1..1836: mol_type = other DNA
atggagatcggaggcgaggaggtggaagtggaagagttcgtggaggaggaggacgaggacacagtggcggcgctgcaggaaaaaaaaagctgcagccagcgcatggcggaatttcgccagtattgctggaacccggataccggccagatgctgggccgcaccccggcgcgctgggtgtggattagcctgtattatgcggcgttttatgtggtgatgaccggcctgtttgcgctgtgcatttatgtgctgatgcagaccattgatccgtataccccggattatcaggatcagctgaaaagcccgggcgtgaccctgcgcccggatgtgtatggcgaacgcggcctgcagattagctataacattagcgaaaacagcagcatggaccccatcgctctgcaggctggttacgacctgctgggtgacggcagacctgaaactctgtggctgggcatcggcactctgctgatgctgattggaaccttctactttctggtccgcggatggggagtcaccgataaggatgcccgggaatattacgctgtgactatcctggtgcccggaatcgcatccgccgcatatctgtctatgttctttggtatcgggcttactgaggtgaccgtcgggggcgaaatgttggatatctattatgccaggtacgccgactggctgtttaccaccccacttctgctgctggatctggcccttctcgctaaggtggatcgggtgaccatcggcaccctggtgggtgtggacgccctgatgatcgtcactggcctcatcggagccttgagccacacggccatagccagatacagttggtggttgttctctacaatttgcatgatagtggtgctctattttctggctacatccctgcgatctgctgcaaaggagcggggccccgaggtggcatctacctttaacaccctgacagctctggtcttggtgctgtggaccgcttaccctatcctgtggatcataggcactgagggcgctggcgtggtgggcctgggcatcgaaactctgctgtttatggtgttggacgtgactgccaaggtcggctttggctttatcctgttgagatcccgggctattctgggcgacaccgaggcaccagaacccagtgccggtgccgatgtcagtgccgccgacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 4 - DNA, 584 bp, synthetic construct
  misc_feature 1..584: note = DNA sequence of a promoter used upstream of 82EO-sequences for targeting neurons or other cells as an example
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg